(19) United States
(12) Patent Application Publication
(10) Pub. No.: US 2008/0118493 A1
(43) Pub. Date: May 22, 2008

(54) Elevated Hedgehog Pathway Activity In Digestive System Tumors, And Methods Of Treating Digestive Sytem Tumors Having Elevated Hedgehog Pathway Activity

(76) Inventors: Philip A. Beachy, Towson, MD (US); David Monty Berman, Towson, MD (US); Sunil S. Karhadkar, Towson, MD (US); Anirban Maitra, Kensington, MD (US)

Correspondence Address: DLA PIPER US LLP, 4365 EXECUTIVE DRIVE, SUITE 1100, SAN DIEGO, CA, 92121-2133, US

(21) Appl. No.: 10/564,580

(22) PCT Filed: Jul. 15, 2004
(86) PCT No.: PCT/US04/22698

§ 371 Date: Dec. 20, 2007

Related U.S. Application Data
(60) Provisional application No. 60/487,554, filed on Jul. 15, 2003.

(51) Int. Cl. A61K 31/395; C12N 5/04; A61K 38/00; A61K 31/70; C12Q 1/20; C12Q 1/68

(52) U.S. Cl. 424/130.1; 435/375; 514/2; 514/44; 435/29; 435/6

(57) ABSTRACT

Elevated Hedgehog (Hh) pathway activity, including ligand stimulated Hh pathway activity, was detected in digestive tract cancers, including esophagus, stomach, biliary tract, and pancreatic cancer, and determined to be associated with growth and proliferation of the cancer cells. Accordingly, methods are provided for treating a digestive tract cancer associated with elevated Hh pathway activity by reducing or inhibiting the Hh pathway activity. Also provided are methods of determining the responsiveness of a digestive tract tumor to treatment with an Hh pathway antagonist.

### CROSS REFERENCE TO RELATED APPLICATION(S)

This application claims the benefit of priority under 35 U.S.C. § 119(e) of U.S. Ser. No. 60/487,554, filed Jul. 15, 2003, the entire content of which is incorporated herein by reference.

### GRANT INFORMATION

This invention was made with government support under Grant Nos. CA57341 and CA62924 awarded by the National Institutes of Health. The United States government has certain rights in this invention.

### BACKGROUND OF THE INVENTION

1. Field of the Invention

The invention relates generally to methods of treating a cancer of the digestive system, and more specifically to methods of reducing or inhibiting proliferation of cancer cells of a digestive tract tumor that is characterized, at least in part, by elevated Hedgehog (Hh) pathway activity as compared to the Hh pathway activity in normal cells of the corresponding organ, and to methods of identifying agents that can be used to treat a subject having a digestive tract tumor characterized by abnormally elevated Hh pathway activity.

2. Background Information

Cancers of the digestive system are a relatively common form of cancer in humans. Due to their nature, digestive system cancers, including, for example, pancreatic cancer and stomach cancer, often are silent, and are not detected until they have reached a relatively advanced stage. As such, digestive tract cancers are associated with substantial morbidity and mortality. Further, the relatively high incidence of digestive system cancers in humans, in combination with the advanced stage at which they generally are detected, results in a significant economic burden both to the patient due to the costs of treatment and to lost wages, and to the economy in general due to loss of the cancer patients from the labor force.

When detected at an early enough stage, digestive tract cancers can be treated by surgery, radiation therapy, chemotherapy, or a combined modality therapy such as surgery to debulk the tumor, followed by chemotherapy to kill remaining tumor cells, including any metastatic disease. Surgery and, in some cases, radiotherapy can provide the advantage that they can reduce the tumor mass, while mostly sparing normal tissues. However, these methods are limited to treating patients with localized disease. In comparison, chemotherapy can be advantageous where the disease has spread, or is not otherwise amenable to surgery or radiotherapy. Of course, the disadvantage of chemotherapy is that it is relatively non-specific and, therefore, kills normal cells, particularly in rapidly renewing tissues such as blood, skin, and the intestine.

In order to develop drugs and methods for specifically treating a cancer, while sparing normal tissues, an understanding of the molecular mechanisms involved in the etiology of the disease is required. For example, by identifying one or more molecular pathways that are aberrantly regulated in a cancer cell as compared to a corresponding normal cell, and further identifying the defect leading to the aberrant regulation, drugs can be developed that target the defect and, therefore, can be relatively specific for the cancer cells having the defect. Unfortunately, only a few molecular defects have been identified in digestive tract cancers, and few, if any, therapeutic regimens that exploit such defects have been described.

### SUMMARY OF THE INVENTION

The present invention is based, in part, on the determination that Hedgehog (Hh) pathway activity is abnormally elevated in digestive system tumor cells as compared to corresponding normal cells of the organ with the tumor, and that agents that decrease the Hh pathway activity inhibits proliferation of digestive system tumor cells. For example, abnormally elevated Hh ligand stimulated Hh pathway activity was detected in tumors originating from esophagus, stomach, biliary tract, and pancreas, and antibodies and small organic molecules that can interfere with ligand stimulated Hh pathway activity inhibited proliferation of the cancer cells. Hh ligands that can stimulate Hh pathway activity include Sonic hedgehog (SHH), Indian hedgehog (IHH), and/or Desert hedgehog (DHH). Abnormally elevated Hh pathway activity also can be due, for example, to a mutation in an Hh ligand receptor such as Patched (PTCH), wherein PTCH in inactivated, resulting in unregulated Smoothened (SMO) activity and elevated Hh pathway activity. Accordingly, the present invention provides methods of treating a digestive tract tumor characterized by abnormally elevated Hh pathway activity, as well as methods of determining whether a digestive tract tumor has such activity and methods of identifying agents useful for treating such tumors. As such, methods of personalized medicine are provided, wherein agents can be selected that are particularly useful for treating a particular digestive tract tumor in a patient.

The present invention relates to a method of reducing or inhibiting proliferation of cells of a digestive tract tumor characterized by abnormally elevated Hh pathway activity. Such a method can be performed, for example, by contacting the cells with at least one (e.g., 1, 2, 3, 4, or more) Hh pathway antagonist, whereby proliferation of the cells of the digestive tract tumor is reduced or inhibited. The Hh pathway generally includes an Hh ligand (e.g., SHH, IHH and/or DHH), which binds an Hh ligand receptor (e.g., PTCH), resulting in activation of SMO (a G protein coupled receptor-like polypeptide), which transduces the Hh signal downstream, resulting in activation of additional members of the Hh pathway (e.g., Fused), including Hh pathway stimulated transcription factors (e.g., members of the GLI family of transcription factors). Also associated with Hh pathway activity are transcriptional targets, including, for example, nestin and BMI-1, which can be induced by activated GLI transcription factor. As such, it will be recognized that an Hh pathway antagonist useful in a method of the invention is selected, in part, in that it acts at or downstream of the position in the Hh pathway associated with the elevated Hh pathway activity. For example, where abnormally elevated Hh pathway activity is ligand stimulated, the Hh antagonist can be selected based on the ability, for example, to sequester the Hh ligand or to reduce or inhibit binding of the Hh ligand to its receptor, or at any point downstream of these events. In comparison, where abnormally elevated Hh pathway activity is due to an inactivating mutation of the Hh ligand receptor (e.g., PTCH), the Hh pathway antagonist can be selected based on the ability, for example, to bind to and inhibit SMO or to reduce the activity of an activating GLI transcription factor (e.g., GLI-1 or GLI-2), but not at a point upstream.

A digestive tract tumor for which cell proliferation can be reduced or inhibited can be any tumor of the digestive system that is characterized, at least in part, by Hh pathway activity that is elevated above levels that are typically found in normal cells corresponding to the tumor cell (e.g., normal esophageal epithelial cells as compared to esophageal adenocarcinoma cells). As such, the digestive tract tumor can be a benign tumor or a malignant tumor, for example, of the mouth, esophagus, stomach, small intestine, large intestine, anus, rectum, gall bladder, or pancreas. Such digestive tract tumors are exemplified herein by pancreatic cancer, stomach cancer, esophageal cancer, and biliary tract cancer, each of which is characterized, in part, by abnormally elevated ligand stimulated Hh pathway activity and increased expression of the Hh ligands Sonic hedgehog (SHH) and/or Indian hedgehog (IHH).

An Hh pathway antagonist useful in a method of the invention can be any antagonist that interferes with Hh pathway activity, thereby decreasing the abnormally elevated Hh pathway in the digestive tract tumor cells. As such, the Hh pathway antagonist can be a peptide, a polynucleotide, a peptidomimetic, a small organic molecule, or any other molecule. Hh pathway antagonists are exemplified by antibodies, including an anti-SHH antibody, an anti-IHH antibody, and an anti-DHH antibody, each of which can bind to at least one Hh ligand and decrease ligand stimulated Hh pathway activity. Hh pathway antagonists useful in the present methods are further exemplified by, but not limited to, steroidal alkaloids and derivatives thereof, including cyclopamine, jervine, and the like, and by the SMO antagonists, SANT-1, SANT-2, SANT-3, and SANT-4.

In one embodiment, the invention relates a method of ameliorating a digestive tract tumor comprising cells characterized by abnormally elevated Hh pathway activity in a subject. Such a method can be performed by administering to the subject at least one Hh pathway antagonist such that the Hh pathway antagonist contacts cells of the tumor in the subject. According to the present method, the Hh pathway antagonist(s) can reduce or inhibit proliferation of the tumor cells, thereby ameliorating the digestive tract tumor in the subject.

A digestive tract tumor in a subject to be treated can be any digestive tract tumor that exhibits abnormally elevated Hh pathway activity (e.g., abnormally elevated ligand stimulated Hh pathway activity). In one aspect, the tumor is a malignant tumor such as a pancreatic cancer, stomach cancer, esophageal cancer, biliary tract cancer, or colon cancer cells. The Hh pathway antagonist(s) can be administered in any way typical of an agent used to treat the particular type of digestive tract tumor. For example, the Hh pathway antagonist(s) can be administered orally or parenterally, including, for example, by injection or as a suppository, or by any combination of such methods.

The Hh pathway antagonist can be any type of compound as disclosed herein or otherwise having the ability to interfere with Hh pathway activity. In one aspect, the Hh pathway antagonist is an antibody, for example, an antibody specific for one or more Hh ligand(s) (e.g., an anti-SHH, anti-IHH, and/or anti-DHH antibody). In another aspect, the Hh pathway antagonist is a SMO antagonist such as a steroidal alkaloid, or a derivative thereof (e.g., cyclopamine or jervine), or other synthetic small molecule such as SANT-1, SANT-2, SANT-3, or SANT-4. In still another aspect, a combination of Hh pathway antagonists are administered to the subject. Further, any additional compounds that can provide a therapeutic benefit can be administered to the subject, including, for example, a chemotherapeutic agent or nutritional supplement, and/or the subject can be further treated, for example, by radiation therapy or using a surgical procedure.

The present invention further relates to a method of identifying a digestive tract tumor of a subject amenable to treatment with a Hh pathway antagonist. As such, the method provides a means to determine whether a subject having a digestive tract tumor, or particular type of digestive tract tumor, is likely to be responsive to treatment with an Hh pathway antagonist. The method can be performed, for example, by detecting abnormally elevated Hh pathway activity in a sample of cells of the digestive tract tumor of the subject as compared to corresponding normal cells, wherein detection of an abnormally elevated level indicates that the subject can benefit from treatment with an Hh pathway antagonist. The sample of cells can be any sample, including, for example, a tumor sample obtained by biopsy of a subject having the tumor or a tumor sample obtained by surgery (e.g., a surgical procedure to remove and/or debulk the tumor). The Hh pathway activity can be abnormally elevated due, for example, to a mutation of a gene encoding an Hh pathway polypeptide (e.g., an inactivating mutation of PTCH), or can be abnormally elevated ligand stimulated Hh pathway activity.

In one embodiment, the method of identifying a digestive tract tumor amenable to treatment with a Hh pathway antagonist includes detecting an abnormal level of expression of one or more Hh pathway polypeptide(s), including, for example, one or more Hh ligands (e.g., SHH, IHH, and/or desert hedgehog), Hh ligand receptors (e.g., PTCH), or transcription factors (a GLI family member). In one aspect, the abnormal expression is an abnormally elevated expression of one or more Hh pathway polypeptide(s), including, for example, one or more Hh ligands (e.g., SHH, IHH, and/or desert hedgehog), Hh ligand receptors (e.g., PTCH), or transcription factors (a GLI family member), or a combination of such Hh pathway polypeptides. In another aspect of this embodiment, the abnormal level of expression is an abnormally low expression of one or more Hh pathway polypeptide(s), including, for example, GLI-3, which acts as a transcriptional repressor in the Hh pathway. Increased or decreased expression of an Hh pathway polypeptide can be detected by measuring the level of a polynucleotide encoding the Hh pathway polypeptide using, for example, a hybridization assay, a primer extension assay, or a polymerase chain reaction assay (e.g., measuring the level of PTCH mRNA expression and/or GLI mRNA expression); or by measuring the level the Hh pathway polypeptide(s) using, for example, an immunoassay or receptor binding assay

In another embodiment, the method of identifying a digestive tract tumor amenable to treatment with a Hh pathway antagonist includes detecting an abnormally elevated activity of one or more Hh pathway polypeptide(s). For example, abnormally elevated activity of Hh pathway transcription factor (e.g., a GLI family member) can be detected by measuring increased binding activity of the transcription factor to a cognate transcription factor regulatory element (e.g., using an electrophoretic mobility shift assay); by measuring increased expression of a reporter gene comprising a cognate transcription factor regulatory element; or measuring expression of GLI and/or of PTCH, and/or a target of the GLI transcription factor (e.g., by detecting transcription of nestin or BMI-1). In still another embodiment, the method can include detecting expression of an Hh pathway polypeptide having an inactivating mutation, wherein the mutation is associated with abnormally elevated Hh pathway activity (e.g., by detecting expression of a mutant PTCH Hh ligand receptor).

The method of identifying a digestive tract tumor amenable to treatment with a Hh pathway antagonist can further include contacting cells of the sample with at least one Hh pathway antagonist, and detecting a decrease in Hh pathway activity in the cells following said contact. The decreased Hh pathway activity can be detected, for example, by measuring decreased expression of a reporter gene regulated by an Hh pathway transcription factor, or by detecting a decreased in proliferation of the tumor cells. Such a method provides a means to confirm that the digestive tract tumor is amenable to treatment with an Hh pathway antagonist. Further, the method can include testing one or more different Hh pathway antagonists, either alone or in combination, thus providing a means to identify one or more Hh pathway antagonists useful for treating the particular digestive tract tumor being examined.

The present invention further relates to a method of identifying an agent useful for treating a digestive tract tumor having abnormally elevated Hh pathway activity. In one embodiment, the method provides a means for practicing personalized medicine, wherein treatment is tailored to the particular patient based on the characteristics of the digestive tract tumor in the patient. The present method can be practiced, for example, by contacting a sample of cells of a digestive tract tumor with at least one test agent, wherein a decrease in Hh pathway activity in the presence of the test agent as compared to Hh pathway activity in the absence of the test agent identifies the agent as useful for treating the digestive tract tumor. As disclosed herein, abnormally elevated Hh pathway activity can be due to abnormally elevated ligand stimulated Hh pathway activity or to a mutation that results in elevated Hh pathway activity (e.g., an inactivating mutation of PTCH, or a mutation resulting in a constitutively active GLI transcription factor).

The present method can be practiced using test agents that are known to be effective in treating a digestive tract tumor having abnormally elevated Hh pathway activity in order to identify one or more agents that are particularly useful for treating the digestive tract tumor being examined, or using test agents that are being examined for effectiveness. As such, in one aspect, the test agent examined according to the present method can be any type of compound, including, for example, a peptide, a polynucleotide, a peptidomimetic, or a small organic molecule, and can be one of a plurality of similar but different agents (e.g., a combinatorial library of test agents, which can be a randomized or biased library or can be a variegated library based on known effective agent such as the known Hh pathway antagonist, cyclopamine). In another aspect, the test agent comprises a known Hh pathway antagonist such as an antibody (e.g., an anti-SHH antibody and/or anti-IHH antibody) or a steroidal alkaloid or a derivative thereof (e.g., cyclopamine, jervine, or triparanol).

The sample of cells used in the present method can be cells obtained (e.g., by biopsy or other surgical procedure) from a subject having the digestive tract tumor, including primary tumor cells; or can be cells that have been placed in and/or adapted to culture, including, for example, cells of an established digestive tract tumor cell line (or a plurality of such established cell lines, which can provide a panel for examining test agents according to the present method). The digestive tract tumor sample can be a malignant tumor sample such as pancreatic cancer cells, stomach cancer cells, esophagus cancer cells, biliary tract cancer cells, or colon cancer cells. Generally, though not necessarily, the method is performed by contacting the sample of cells ex vivo, for example, in a culture medium or on a solid support. As such, the methods are conveniently adaptable to a high throughput format, wherein a plurality (i.e., 2 or more) of samples of cells, which can be the same or different, are examined in parallel.

A high throughput format provides numerous advantages, including that test agents can be tested on several samples of cells from a single patient, thus allowing, for example, for the identification of a particularly effective concentration of an agent to be administered to the subject, or for the identification of a particularly effective agent to be administered to the subject. As such, a high throughput format allows for the examination of two, three, four, etc., different test agents, alone or in combination, on the cells of a subject's digestive tract tumor such that the best (most effective) agent or combination of agents can be used for a therapeutic procedure. Accordingly, in various embodiments, the high throughput method is practiced by contacting different samples of cells of different subjects with same amounts of a test agent; or contacting different samples of cells of a single subject with different amounts of a test agent; or contacting different samples of cells of two or more different subjects with same or different amounts of different test agents. Further, a high throughput format allows, for example, control samples (positive controls and or negative controls) to be run in parallel with test samples, including, for example, samples of cells known to be effectively treated with an agent being tested. Variations of the exemplified methods also are contemplated.

### BRIEF DESCRIPTION OF THE DRAWINGS

FIG. 1 shows the widespread expression of transcripts encoding hedgehog (Hh) pathway components in digestive tract tumor cell lines. RT-PCR products demonstrating expression of genes encoding Hh pathway ligands, Sonic hedgehog and Indian hedgehog (SHH and IHH) and target genes, PTCH and GLI in tumor cell lines from sites in diagram (left). Red bars (right) indicate the percent of tumor cell lines expressing detectable PTCH mRNA at each site.

FIGS. 2A and 2B demonstrate that cyclopamine suppression of Hh pathway activity and growth in digestive tract tumor cell lines correlates with expression of PTCH mRNA.

FIG. 2A shows normalized activity of transiently transfected Hh-responsive luciferase reporter and dose-dependent suppression by the Hh pathway antagonist cyclopamine.

FIG. 2B shows the change in tumor cell viability measured by MTS (soluble tetrazolium salt) assay after culture in 3.0 μM cyclopamine or tomatidine (control). (“Bil” indicates biliary).

FIGS. 3A to 3D demonstrate the Hh pathway activity and requirement for growth of tumor cells in vivo.

FIG. 3A reveals elevated PTCH mRNA in surgically resected pancreatic and gastric carcinomas, as detected by quantitative RT-PCR and normalized to adjacent normal stomach (n=10) and pancreas (n=1).

FIG. 3B shows normalized Hh-responsive reporter activity and suppression by 3.0 μM cyclopamine in first passage pancreas carcinoma xenografts.

FIG. 3C shows a corresponding reduction in viable tumor cells upon culture with 3.0 μM cyclopamine. Reduced viability is observed exclusively in xenograft lines with elevated Hh pathway activity.

FIG. 3D shows the change in human HuCCT1 human cholangiocarcinoma xenograft tumor volume in mice treated for 14 days with vehicle (control; n=9) or cyclopamine (n=9).

FIGS. 4A to 4F demonstrate the ligand dependence of Hh pathway activity and growth in digestive tract tumors.

FIG. 4A shows the mutually antagonistic effects of Hh ligand and blocking antibody on activity of a Hh reporter. The Hh neutralizing 5E1 monoclonal antibody suppresses and Sonic hedgehog (Shh) ligand increases reporter activity in HuCCT1 cells. Combined addition of antibody and ligand produces intermediate effects, depending on relative concentrations.

FIG. 4B shows Hh reporter activity in first passage pancreas carcinoma xenografts and dose-dependent suppression with 5E1 MAb.

FIG. 4C provides an MTS assay demonstrating reduced viability corresponding to Hh pathway suppression by 5E1 MAb.

FIG. 4D provides an MTS assay showing growth (in arbitrary units) of PX 184 first passage PTCH mRNA expressing pancreas xenograft cells cultured in control antibody (dashed line) or with 5E1 MAb at a level just sufficient to suppress growth (0.1 μg/ml; solid lines), and with the indicated concentrations of added Shh ligand.

FIG. 4E shows the growth rate (in arbitrary units) of PX184 cells (obtained using date of FIG. 4D). Dashed line represents growth rate of cells cultured with control antibody.

FIG. 4F demonstrates the modulation of cell growth rate by 5E1 MAb and Shh ligand in single passage pancreatic xenografts (PX-184, PX169) and medulloblastoma cells (PZp53^{MED1}). Note opposite responses to ligand and antibody of PX-184 cells, which express PTCH mRNA, and the lack of response of PX-169 and PZp53^{MED1} cells, which respectively lack detectable Hh pathway activation, or display constitutive pathway activation due to lack of functional PTCH (see Berman et al., Science 297, 1559-1561, 2002, which is incorporated herein by reference).

### DETAILED DESCRIPTION OF THE INVENTION

The present invention is based on the identification of elevated hedgehog (Hh) pathway activity in tumors derived from the gut, a tissue with prominent and diverse roles for Hh signaling in developmental patterning and tissue homeostasis (see Berman et al., Nature 425:846-851, 2003, which is incorporated herein by reference; see, also, Refs. 8-10, below). Activation of the Hh signaling pathway by sporadic mutations or in familial conditions such as Gorlin syndrome has been associated with tumorigenesis in skin, cerebellum, and skeletal muscle (see Refs. 1, 2). As disclosed herein, a wide range of digestive tract tumors, including the majority of those originating from esophagus, stomach, biliary tract, and pancreas, displayed elevated levels of Hh pathway activity that were suppressed by the Hh pathway antagonist cyclopamine (see Example 1). Cyclopamine also suppressed cell growth in vitro and caused regression of xenograft tumors in vivo. Unlike Gorlin syndrome tumors, Hh pathway activity and cell growth in a variety of digestive tract tumors was driven by endogenous expression of Hh ligands, as indicated by the presence of Sonic hedgehog (SHH) and Indian hedgehog (IHH) transcripts, by the pathway-inhibitory and growth-inhibitory activity of an Hh-neutralizing antibody, and by the dramatic growth-stimulatory activity of exogenously added Hh ligand. These results demonstrate that a group of common lethal malignancies are characterized by abnormally elevated Hh pathway activity that is essential for tumor growth. Accordingly, the present invention provides methods of treating a digestive tract tumor characterized by abnormally elevated Hh pathway activity, as well as methods of determining whether a digestive tract tumor is amenable to treatment using an Hh pathway antagonist, and methods of identifying agents useful for treating such tumors.

As used herein, reference to the “Hh pathway” means the Hedgehog signal transduction pathway. The Hh pathway is well known (see, e.g., U.S. Pat. No. 6,277,566 B1; U.S. Pat. No. 6,432,970 B2; Lum and Beachy, Science 304:1755-1759, 2004; and Bale and Yu, Hum. Mol. Genet. 10:757-762, 2001, each of which is incorporated herein by reference). Briefly, SHH, IHH and DHH are a family of secreted proteins that act as ligand (Hh ligands) to initiate the Hh pathway, which is involved in morphogenetic development and proliferation of cells in a variety of tissues. Hh ligands bind to a receptor complex that includes Patched (PTCH; e.g., PTCH-1 in humans) and Smoothened (SMO), which a G-protein coupled receptor-like polypeptide. PTCH is an integral membrane protein with twelve transmembrane domains that acts as an inhibitor of SMO activation. Hh ligand binding to PTCH results in activation of SMO (see, e.g., Taipale et al., Nature 418:892-897, 2002, which is incorporated herein by reference), resulting in transduction of the signal and activation of the GLI family of transcriptional activators (e.g., GLI-1 and GLI-2, which act as transcriptional activators, and GLI-3, which acts as a transcriptional repressor), which are homologs of the Drosophila cubitis interruptis gene. Several kinases also are believed to be involved in the Hh pathway between SMO and the GLI transcription factors, including, for example, protein kinase A, which can inhibit GLI activity. Suppressor of Fused (SUFU) also interacts directly with GLI transcription factors to repress their activity. In addition, various transcriptional targets such as nestin and BMI-1 are regulated by Hh pathway activity.

The Hedgehog (Hh) signaling pathway specifies patterns of cell growth and differentiation in a wide variety of embryonic tissues. Mutational activation of the Hh pathway, whether sporadic or in Gorlin Syndrome, is associated with tumorigenesis in a limited subset of these tissues, predominantly skin, cerebellum, and skeletal muscle^{1,2}. Known pathway-activating mutations include those that impair the ability of PTCH (the target of Gorlin Syndrome mutations), a transporter-like Hh receptor³, to restrain Smoothened (SMO) activation of transcriptional targets via the GLI family of latent transcription factors (see Refs. 1, 2, 4, 5). Binding of Hh ligand to PTCH is functionally equivalent to genetic loss of PTCH, in that pathway activation by either requires activity of SMO, a seven transmembrane protein that binds to and is inactivated by the pathway antagonist, cyclopamine⁶.

The term “Hh pathway activity” is used herein to refer to the level of Hedgehog pathway signal transduction that is occurring in cells. Hh pathway activity can be determined using methods as disclosed herein (see Example 1) or otherwise known in the art (see, e.g., Refs. 14 and 22). As used herein, the term “abnormally elevated”, when used in reference to Hh pathway activity, means that the Hh pathway activity is increased above the level typically found in normal (i.e., not cancer) differentiated cells of the same type as the cells from which the tumor are derived. As such, the term “abnormally elevated Hh pathway activity” refers to the level of Hh pathway activity in digestive tract tumor cells as compared to corresponding normal cells. Generally, abnormally elevated Hh pathway activity is at least about 20% (e.g., 30%, 40%, 50%, 60%, 70%, or more) greater than the Hh pathway activity in corresponding normal cells. In this respect, it should be recognized that Hh pathway activity is determined with respect to a population of cells, which can be a population of tumor cells or a population of normal cells, and, therefore, is an average activity determined from the sampled population.

Reference herein to “corresponding normal cells” means cells that are from the same organ and of the same type as the digestive tract tumor cell type. For example, with respect to a pancreatic ductal adenocarcinoma cell, a corresponding normal cell would be a pancreatic ductal epithelial cell that is not a cancer cell. In one aspect, the corresponding normal cells comprise a sample of cells obtained from a healthy individual. Such corresponding normal cells can, but need not be, from an individual that is age-matched and/or of the same sex as individual providing the digestive tract tumor cells being examined. In another aspect, the corresponding normal cells comprise a sample of cells obtained from an otherwise healthy portion of tissue of a subject having a digestive tract tumor.

The invention provides methods of reducing or inhibiting Hh pathway activity and/or proliferation of cells of a digestive tract tumor characterized by abnormally elevated Hh pathway activity. As used herein, the terms “reduce” and “inhibit” are used together because it is recognized that, in some cases, a decrease, for example, in Hh pathway activity can be reduced below the level of detection of a particular assay. As such, it may not always be clear whether the activity is “reduced” below a level of detection of an assay, or is completely “inhibited”. Nevertheless, it will be clearly determinable, following a treatment according to the present methods, that the level of Hh pathway activity (and/or cell proliferation) is at least reduced from the level before treatment. Generally, contact of digestive tract tumor cells having abnormally elevated Hh pathway activity with an Hh pathway antagonist reduces the Hh pathway activity by at least about 20% (e.g., 30%, 40%, 50%, 60%, 70%, or more). For example, the Hh pathway activity in a digestive tract tumor cell treated according to the present methods can be reduced to the level of Hh pathway activity typical of a corresponding normal cell.

An Hh pathway antagonist useful in a method of the invention generally acts at or downstream of the position in the Hh pathway that is associated with the elevated Hh pathway activity. For example, where abnormally elevated Hh pathway activity is ligand stimulated, the Hh antagonist can be selected based on the ability, for example, to sequester the Hh ligand (e.g., an antibody specific for the Hh ligand) or to reduce or inhibit binding of the Hh ligand to its receptor. Since Hh ligand activity is dependent, on autoprocessing of the Hh ligand (e.g., SHH) into a C-terminal fragment, and an N-terminal fragment that is further modified by attachment of cholesterol and palmitate molecules (and constitutes the ligand; see, e.g., Mann and Beachy, Ann. Rev. Biochem. 73:891-923, 2004, which is incorporated herein by reference), ligand stimulated Hh pathway activity also can be reduced or inhibited by inhibiting autocleavage of the Hh ligand. Where abnormally elevated Hh pathway activity is due to an inactivating mutation of the Hh ligand receptor (e.g., PTCH), the Hh pathway antagonist can be selected based on the ability, for example, to sequester SMO (e.g., an antibody specific for SMO) or to reduce activity of a GLI transcription factor (e.g., a polynucleotide comprising a GLI regulatory element, which can act to sequester GLI); an anti-Hh ligand antibody may not necessarily reduce or inhibit elevated Hh pathway activity due to a mutation of PTCH because Hh ligand acts upstream of the defect in the Hh pathway. Further, steroidal alkaloids, and derivatives thereof, and other small molecules such as SANT-1, SANT-2, SANT-3, and SANT-4 can reduce or inhibit abnormally elevated Hh pathway activity by directly repressing SMO activity. In addition, cholesterol can be required for Hh pathway activity and, therefore, agents that reduce the availability of cholesterol, for example, by removing it from cell membranes, can act as Hh pathway antagonists (see, e.g., Cooper et al., Nat. Genet 33:508-513, 2003, which is incorporated herein by reference; see, also, Cooper et al., Nat. Genet. 34:113, 2003).

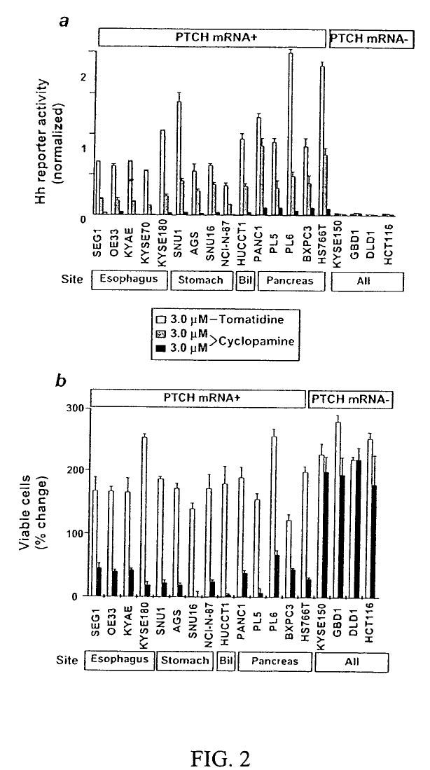

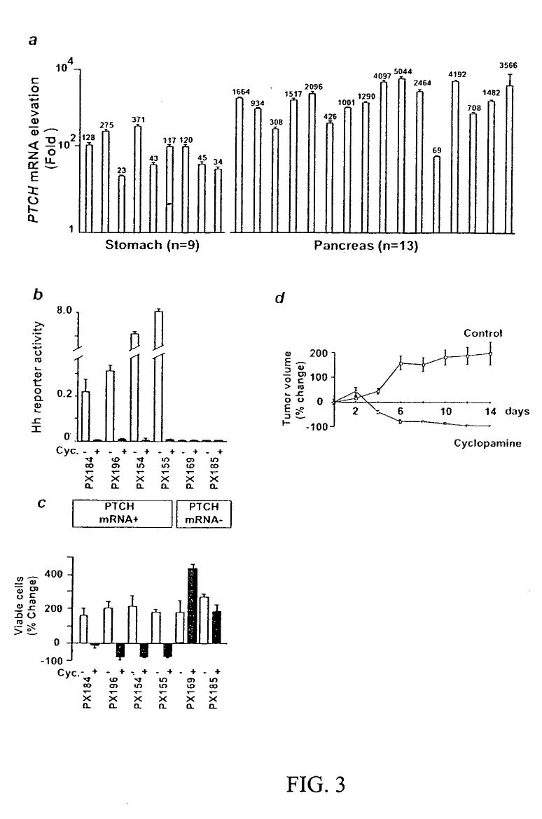

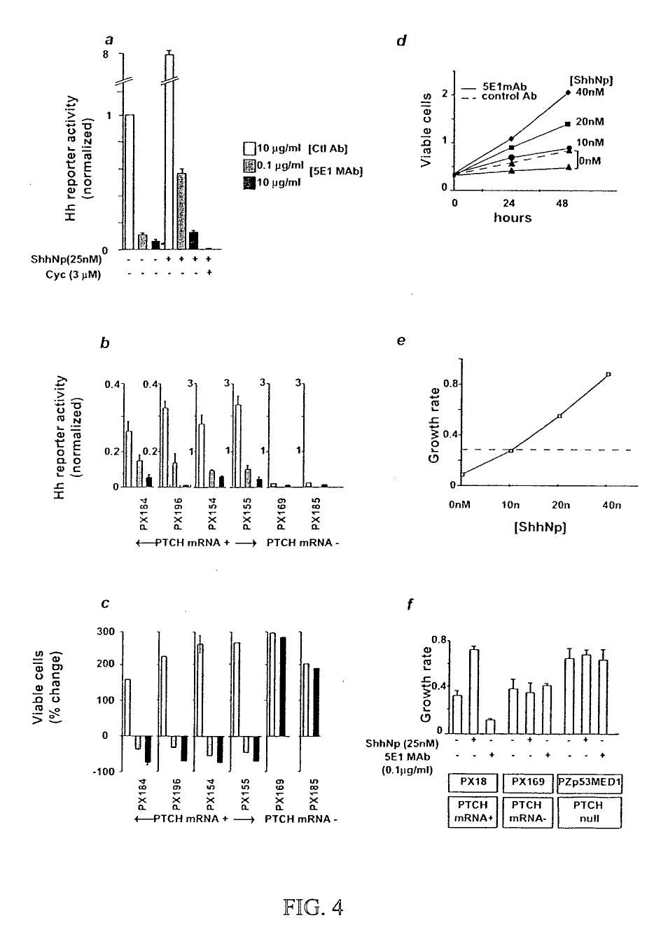

An Hh pathway antagonist useful in a method of the invention can be any antagonist that interferes with Hh pathway activity, thereby decreasing the abnormally elevated Hh pathway in the digestive tract tumor cells. As such, the Hh pathway antagonist can be a peptide, a polynucleotide, a peptidomimetic, a small organic molecule, or any other molecule. Hh pathway antagonists are exemplified by antibodies, including anti-SHH antibodies, anti-IHH antibodies, and/or anti-DHH antibodies, each of which can bind to one or more Hh ligands and decrease ligand stimulated Hh pathway activity. Hh pathway antagonists are further exemplified by SMO antagonists such as steroidal alkaloids and derivatives thereof, including, for example, cyclopamine and jervine (see, e.g., Chen et al., Genes Devel. 16:2743-2748, 2002; and U.S. Pat. No. 6,432,970 B2, each of which is incorporated herein by reference), and SANT-1, SANT-2, SANT-3, and SANT-4 (see Chen et al., Proc. Natl. Acad. Sci., USA 99:14071-14076, 2002, which is incorporated herein by reference); triparanol provides another example of an agent that can act as an Hh pathway antagonist (see, e.g., U.S. Pat. No. 6,432,970 B2). As exemplified herein, an anti-SHH antibody and cyclopamine effectively reduced abnormally elevated Hh pathway activity in a variety of digestive tract tumor cells and reduced viability of the cells in vitro (see, e.g., FIGS. 2A and 2B, and FIG. 4A), and cyclopamine suppressed growth of pancreatic tumor xenografts in nude mice (see FIG. 3D).

In one aspect, the present invention provides a method of ameliorating a digestive tract tumor comprising cells characterized by abnormally elevated Hh pathway activity in a subject. As used herein, the term “ameliorate” means that the clinical signs and/or the symptoms associated with the digestive tract tumor are lessened. The signs or symptoms to be monitored will be characteristic of a particular digestive tract tumor and will be well known to skilled clinician, as will the methods for monitoring the signs and conditions. For example, the skilled clinician will know that the size or rate of growth of a tumor can monitored using a diagnostic imaging method typically used for the particular digestive tract tumor (e.g., using ultrasound or magnetic resonance image (MRI) to monitor a pancreatic tumor).

A digestive tract tumor for which Hh pathway activity and cell proliferation can be reduced or inhibited can be any tumor of the digestive system that is characterized, at least in part, by Hh pathway activity that is elevated above levels that are typically found in a normal cell corresponding to the tumor cell (e.g., normal gall bladder or bile duct epithelial cells as compared to gall bladder or bile duct adenocarcinoma cells, respectively). As such, the digestive tract tumor, which can be a benign tumor (e.g., an adenoma such as a polyp) or can be a malignant tumor (e.g., an adenocarcinoma or squamous cell carcinoma), can be a tumor of any portion of the digestive tract, including, for example, the lips, mouth (e.g., oral mucosa epithelium, or salivary glands), pharynx, esophagus, stomach, small intestine, large intestine, anal-rectal region, gall bladder, or pancreas. Such digestive tract tumors are exemplified herein by pancreatic cancer, stomach cancer, esophageal cancer, and biliary tract cancer, each of which is characterized, in part, by abnormally elevated ligand stimulated Hh pathway activity and increased expression of the Hh ligands SHH and/or IHH (see Example 1).

An agent useful in a method of the invention can be any type of molecule, for example, a polynucleotide, a peptide, a peptidomimetic, peptoids such as vinylogous peptoids, a small organic molecule, or the like, and can act in any of various ways to reduce or inhibit abnormally elevated Hh pathway activity. Further, the agent (e.g., an Hh pathway antagonist) can be administered in any way typical of an agent used to treat the particular type of digestive tract tumor or under conditions that facilitate contact of the agent with the target tumor cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell.

An agent useful in a method of the invention can be administered to the site of the digestive tract tumor, or can be administered by any method that results in the agent contacting the target tumor cells. Generally, the agent generally is formulated in a composition (e.g., a pharmaceutical composition) suitable for administration to the subject, which can be any vertebrate subject, including a mammalian subject (e.g., a human subject). Such formulated agents are useful as medicaments for treating a subject suffering from a digestive tract tumor that is characterized, in part, by abnormally elevated Hh pathway activity.

Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The agent, which acts as an Hh pathway antagonist to reduce or inhibit the abnormally elevated Hh pathway activity, can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest. 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of a composition containing the Hh pathway antagonist will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the agent is a small organic molecule such as a steroidal alkaloid (e.g., cyclopamine), it can be administered in a form that releases the active agent at the desired position in the digestive tract (e.g., the stomach), or by injection into a blood vessel that the agent circulates to the target cells (e.g., pancreas).

A composition containing an Hh pathway antagonist can be administered to an individual by various routes including, for example, orally or parenterally, such as intravenously, intramuscularly, subcutaneously, intraperitoneally, intrarectally, intracisternally or, if appropriate, by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. As mentioned above, the pharmaceutical composition also can be administered to the site of digestive tract tumor, for example, intravenously or intra-arterially into a blood vessel supplying a tumor.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the Hh pathway antagonist to treat a digestive tract tumor in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

The pharmaceutical composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The invention also provides a method of determining whether a digestive tract tumor of a subject is amenable to treatment with a Hh pathway antagonist as disclosed herein. The method can be performed, for example, by measuring the level Hh pathway activity in a digestive tract tumor cell sample of the tumor of a subject to be treated, and determining that Hh pathway activity is abnormally elevated as compared to the level of Hh pathway activity in corresponding normal cells, which can be a sample of normal (i.e., not tumor) cells of the subject having the tumor. Detection of abnormally elevated level Hh pathway activity in the tumor cells as compared to the corresponding normal cells indicates that the subject can benefit from treatment with an Hh pathway antagonist. A sample of cells used in the present method can be obtained using a biopsy procedure (e.g., a needle biopsy), or can be a sample of cells obtained by a surgical procedure to remove and/or debulk the tumor.

Abnormally elevated Hh pathway activity can be determined by measuring abnormally elevated expression of one or more (e.g., 1, 2, 3, or more) Hh pathway polypeptide(s), including, for example, one or more Hh ligands (e.g., SHH, IHH, and/or desert hedgehog), Hh ligand receptors (e.g., PTCH), or transcription factors (a GLI family member), or a combination of such Hh pathway polypeptides. The abnormally elevated expression can be detected by measuring the level of a polynucleotide encoding the Hh pathway polypeptide (e.g., RNA) using, for example, a hybridization assay, a primer extension assay, or a polymerase chain reaction (PCR) assay (e.g., a reverse transcription-PCR assay; see Example 1); or by measuring the level the Hh pathway polypeptide(s) using, for example, an immunoassay or receptor binding assay. Alternatively, or in addition, abnormally elevated activity of one or more (e.g., 1, 2, 3, or more) Hh pathway polypeptide(s) can be determined. For example, abnormally elevated activity of Hh pathway transcription factor (e.g., a GLI family member) can be detected by measuring increased binding activity of the transcription factor to a cognate transcription factor regulatory element (e.g., using an electrophoretic mobility shift assay), or by measuring increased expression of a reporter gene comprising a cognate transcription factor regulatory element. Expression of an Hh pathway polypeptide having an inactivating mutation can be identified using, for example, an antibody that specifically binds to the mutant, but not to the normal (wild type), Hh polypeptide, wherein the mutation is associated with abnormally elevated Hh pathway activity. For example, common mutations that result in expression of an inactivated PTCH can define unique epitopes that can be targeted by diagnostic antibodies that specifically bind the mutant, but not wild type, PTCH protein.

The method of identifying a digestive tract tumor amenable to treatment with a Hh pathway antagonist can further include contacting cells of the sample with at least one Hh pathway antagonist, and detecting a decrease in Hh pathway activity in the cells following said contact. The decreased Hh pathway activity can be detected, for example, by measuring decreased expression of a reporter gene regulated by an Hh pathway transcription factor, or by detecting a decreased in proliferation of the tumor cells. Such a method provides a means to confirm that the digestive tract tumor is amenable to treatment with an Hh pathway antagonist. Further, the method can include testing one or more different Hh pathway antagonists, either alone or in combination, thus providing a means to identify one or more Hh pathway antagonists useful for treating the particular digestive tract tumor being examined. Accordingly, the present invention also provides a method of identifying an agent useful for treating a digestive tract tumor having abnormally elevated Hh pathway activity.

The method of identifying an agent useful for treating a digestive tract tumor provides a means for practicing personalized medicine, wherein treatment is tailored to a patient based on the particular characteristics of the digestive tract tumor in the patient. The method can be practiced, for example, by contacting a sample of cells of a digestive tract tumor with at least one test agent, wherein a decrease in Hh pathway activity in the presence of the test agent as compared to Hh pathway activity in the absence of the test agent identifies the agent as useful for treating the digestive tract tumor. The sample of cells examined according to the present method can be obtained from the subject to be treated, or can be cells of an established digestive tract tumor cell line of the same type of tumor as that of the patient. In one aspect, the established digestive tract tumor cell line can be one of a panel of such cell lines, wherein the panel can include different cell lines of the same type of tumor and/or different cell lines of different tumors. Such a panel of cell lines can be useful, for example, to practice the present method when only a small number of tumor cells can be obtained from the subject to be treated, thus providing a surrogate sample of the subject's tumor, and also can be useful to include as control samples in practicing the present methods.

The present methods can be practiced using test agents that are known to be effective in treating a digestive tract tumor having abnormally elevated Hh pathway activity (e.g., a steroidal alkaloid such as cyclopamine or jervine; and/or other SMO antagonist such as SANT-1 or SANT-2; and/or an anti-Hh ligand antibody such as an anti-SHH antibody) in order to identify one or more agents that are particularly useful for treating the digestive tract tumor being examined, or using test agents that are being examined for effectiveness. In addition, the test agent(s) examined according to the present method can be any type of compound, including, for example, a peptide, a polynucleotide, a peptidomimetic, or a small organic molecule, and can be one or a plurality of similar but different agents such as a combinatorial library of test agents, which can be a randomized or biased library or can be a variegated library based on known effective agent such as the known Hh pathway antagonist, cyclopamine (see, for example, U.S. Pat. No. 5,264,563; and U.S. Pat. No. 5,571,698, each of which is incorporated herein by reference). Methods for preparing a combinatorial library of molecules, which can be tested for Hh pathway antagonist activity, are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., supra, 1995; a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference; each of which is incorporated herein by reference); an oligosaccharide library (York et al., Carb. Res. 285:99-128, 1996; Liang et al., Science 274:1520-1522, 1996; Ding et al., Adv. Expt. Med. Biol. 376:261-269, 1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents (Gordon et al., J. Med. Chem. 37:1385-1401, 1994; Ecker and Crooke, supra, 1995; each of which is incorporated herein by reference).

The method of identifying an agent useful for treating a digestive tract tumor having abnormally elevated Hh pathway activity can performed by contacting the sample of cells ex vivo, for example, in a culture medium or on a solid support. Alternatively, or in addition, the method can be performed in vivo, for example, by transplanting a tumor cell sample into a test animal (e.g., a nude mouse), and administering the test agent to the test animal (see Example 1). An advantage of the in vivo assay is that the effectiveness of a test agent can be evaluated in a living animal, thus more closely mimicking the clinical situation. Since in vivo assays generally are more expensive, the can be particularly useful as a secondary screen, following the identification of “lead” agents using an in vitro method.

When practiced as an in vitro assay, the methods can be adapted to a high throughput format, thus allowing the examination of a plurality (i.e., 2, 3, 4, or more) of cell samples and/or test agents, which independently can be the same or different, in parallel. A high throughput format provides numerous advantages, including that test agents can be tested on several samples of cells from a single patient, thus allowing, for example, for the identification of a particularly effective concentration of an agent to be administered to the subject, or for the identification of a particularly effective agent to be administered to the subject. As such, a high throughput format allows for the examination of two, three, four, etc., different test agents, alone or in combination, on the cells of a subject's digestive tract tumor such that the best (most effective) agent or combination of agents can be used for a therapeutic procedure. Further, a high throughput format allows, for example, control samples (positive controls and or negative controls) to be run in parallel with test samples, including, for example, samples of cells known to be effectively treated with an agent being tested.

A high throughput method of the invention can be practiced in any of a variety of ways. For example, different samples of cells obtained from different subjects can be examined, in parallel, with same or different amounts of one or a plurality of test agent(s); or two or more samples of cells obtained from one subject can be examined with same or different amounts of one or a plurality of test agent. In addition, cell samples, which can be of the same or different subjects, can be examined using combinations of test agents and/or known effective agents. Variations of these exemplified formats also can be used to identifying an agent or combination of agents useful for treating a digestive tract tumor having abnormally elevated Hh pathway activity.

When performed in a high throughput (or ultra-high throughput) format, the method can be performed on a solid support (e.g., a microtiter plate, a silicon wafer, or a glass slide), wherein samples to be contacted with a test agent are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for Hh pathway activity and/or cell viability.

The following examples are intended to illustrate but not limit the invention.

### EXAMPLE 1

### Ligand Stimulated Hedgehog Pathway Activity is Associated with Growth of Digestive Tumors

This example demonstrates that digestive tract tumors, including esophagus, stomach, biliary tract, and pancreas cancers, display elevated Hh pathway activity, and that cyclopamine, and Hh pathway antagonist, can decrease the elevated Hh pathway activity and inhibit proliferation of the digestive tract cancer cells.

Cells and tissues: Origins and sources of cells and tissues are described in the Table (below). First passage pancreatic cancer xenografts were derived from freshly harvested pancreaticoduodenectomy specimens as described¹⁴. The ability of these xenografts to represent pancreatic tumors in the general population is confirmed by experiments demonstrating that approximately 65% of specimens yielded xenografts. After reaching 25 mm in greatest dimension, xenograft tumors were harvested, minced, and plated into tissue culture vessels in RPMI, 20% Fetal Bovine Serum (FBS) for assays as described below. The diagnosis of frozen samples from gastric and pancreatic adenocarcinoma resections and adjacent normal stomach and pancreas was microscopically confirmed by two pathologists, and RNA was prepared as described¹⁴.

RT-PCR: Total RNA was prepared from frozen sections or from tissue culture monolayers using RNAwiz™ reagent (Ambion, Inc.; Austin Tex.), according to the manufacturer's instructions. cDNA was synthesized from 1 μg of total RNA in a 33 μl reaction using You-Prime™ First-Strand beads (Amersham Pharmacia; Piscataway N.J.) and random hexamers. PCR reactions were performed using 10% of the first strand reaction and oligonucleotide primers specific for the cDNAs of interest for 38 cycles of 1 min, each at 94° C., 55° C., and 72° C. followed by a single 15 min incubation at 72° C. For all primer pairs, specificity was confirmed by sequencing of PCR products. For quantitative RT-PCR, 10% of the first strand reaction was amplified using IQ™-SYBR® Green Supermix reagent, an iCycler IQ™ real time detection system (BioRad; Hercules Calif.) and specific oligonucleotide primers for PTCH or PGK. Amplification was performed at 95° C. for 5 minutes followed by 40 cycles of 10, 15, and 30 seconds at 95° C., 55° C. and 75° C. respectively. Bio-Rad software was used to calculate threshold cycle (CT) values for PTCH and for the housekeeping gene, phosphoglycerate kinase (PGK). For each sample, PTCH expression was derived from the ratio of PTCH to PGK levels using the formula 2^{-ΔCT} where ΔC_{T}=C_{T}-PTCH-C_{T}-PGK. PTCH levels in tumors were presented as a ratio to levels detected in adjacent normal tissue (FIG. 3A).

Hh-responsive reporter assays: Hh-responsive firefly luciferase and control SV-40 Renilla luciferase reporter assays were performed on subconfluent triplicate cultures as described²². Two days after transfection, cells were cultured for two days in assay media: RPMI-1640 (Bio-Whittaker; Walkersville Md.) supplemented with 0.5% (established cell lines) or 20% (first passage xenografts) fetal bovine serum (FBS) and containing combinations of 5E1 anti-Hh monoclonal antibody, recombinant doubly lipid modified Sonic hedgehog (ShhNp) peptide¹², cyclopamine purified from Veratrum extract, or tomatidine (ICN Pharmaceuticals; Costa Mesa Calif.) at the indicated concentrations.

Proliferation assays: Cells were cultured in triplicate in 96 well plates in assay media to which 5E1 MAb, ShhNp, and/or cyclopamine was added at 0 hr, at the indicated concentrations. Viable cell mass was determined by optical density measurements at 490 nm (O.D.490) at 2 and 4 days using the CellTiter96® colorimetric assay (Promega; Madison Wis.). Relative growth was calculated as {OD (day 4)-OD (day 2)}/OD (day 2).

Xenograft treatment: HUCCT1 tumors (n=18) were grown in athymic (nude) mice to 180 mm³ and treated with cyclopamine (50 mg/kg/day, subcutaneous injection) or control vehicle as described¹⁴.

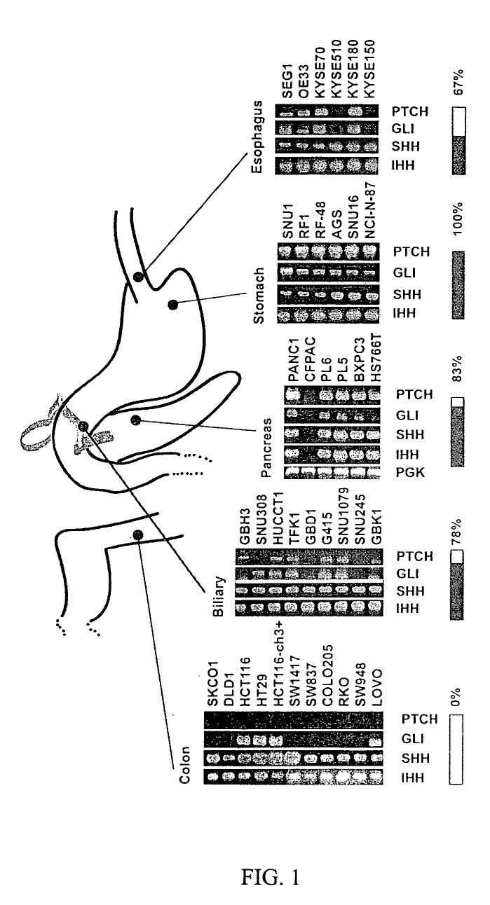

Gut-derived tumors were examined by assaying for expression of Sonic hedgehog (SHH) and Indian hedgehog (IHH), which encode members of the Hh ligand family that are expressed in early endoderm and throughout gut development^{9,11}. SHH and IHH mRNA was detected in 37 of 38 cell lines (97%) from esophagus, stomach, biliary tract, pancreas, and colon carcinomas (see FIG. 1). The Hh target genes PTCH and GLI were co-expressed in most cell lines from esophagus (4/6), stomach (6/6), pancreas (5/6), and biliary tract (5/9) tumors, but not in those derived from colon (0/11). The expression of pathway targets in cells that also express Hh ligands suggests the autonomous operation of an active signaling process within several types of digestive tract tumors.

Autonomous pathway activity was confirmed by the high-level expression of luciferase activity from an exogenously introduced Hh-inducible reporter¹² in all cell lines producing detectable PTCH mRNA (FIG. 2A). Hh pathway activity in these cell lines was inhibited in a dose-dependent manner by the Hh pathway-specific antagonist cyclopamine, but not by tomatidine, an inactive but structurally related compound (FIG. 2A)¹³. These results indicate that high levels of Hh pathway activity may be a common feature of digestive tract tumors. Accordingly, a role for the Hh pathway in tumor growth was investigated. Cyclopamine treatment inhibited growth of tumor cell lines from esophagus, stomach, biliary tract, and pancreas by 75 to 95% as compared to tomatidine controls (FIG. 2B). Significant growth inhibition was observed only in tumor lines expressing PTCH mRNA. These results indicate that the effect of cyclopamine treatment was Hh pathway specific and not due to general cytotoxicity.

Because the properties of cell lines adapted to long term growth in vitro do not always accurately reflect those of tumors growing in vivo, pathway activation was examined in freshly resected stomach and pancreatic tumors by measuring endogenous PTCH mRNA levels. For each specimen, RNA for quantitative RT-PCR analysis was isolated from ten consecutive 10 μM cryosections, after histologic analysis of both immediately flanking sections to determine tumor content. As compared to adjacent normal tissue, PTCH mRNA levels were elevated 23-371 fold in stomach tumors (average=129; n=9) and 69-5044 fold in pancreatic tumors (average=448; n=15; see FIG. 3A).

To examine the role of Hh pathway activity in growth, pancreatic carcinomas were passaged once as xenografts in nude mice, then cultured and immediately assayed in vitro. Of six such xenografts four expressed PTCH mRNA, including a matched pair of primary and metastatic tumors from a single patient. All four of these PTCH-expressing primary xenografts expressed GLI reporter in a cyclopamine-sensitive manner (FIG. 3B). Cyclopamine treatment of these PTCH mRNA-expressing xenografts also resulted in decreased viable cell mass (FIG. 3C), demonstrating more extreme cell-killing effects of Hh pathway blockade than observed in established tumor cell lines (see FIG. 2B). In contrast, single passage xenografts lacking PTCH mRNA grew equally well in control and cyclopamine containing media (FIG. 3C), again confirming that cyclopamine effects were pathway specific rather than generally cytotoxic.

To examine the effects of cyclopamine treatment in vivo, subcutaneous xenografts were established from HuCCT1, a metastatic cholangiocarcinoma cell line. After growth to an average size of 180 mm³, mice bearing these tumors were injected daily with cyclopamine. Complete or near complete regression of all nine treated tumors was observed within 14 days (FIG. 3D; see, also, Berman et al., supra, 2003). Control vehicle-treated tumors, in contrast, continued growing. Consistent with previous reports^{7,14}, all mice survived cyclopamine treatment without obvious adverse reactions. These results demonstrate specific in vivo tumoricidal effects of Hh pathway blockade by treatment with cyclopamine.

Together, the above results demonstrate widespread activation of the Hh pathway in gut-derived tumors, and a role for pathway activity in tumor cell growth in vitro and in vivo. Gorlin syndrome is not associated with a higher incidence of gut-derived tumors, and PTCH mutations in these tumors have not been reported, suggesting a non-mutational mechanism for pathway activation. In view of the observed expression of SHH and IHH mRNA in nearly all gut-derived tumors examined, the role of Hh ligand binding in pathway activity was investigated. Hh-inducible reporter activity was measured in HuCCT1 cholangiocarcinoma cells treated with 5E1 monoclonal antibody¹⁵, which binds SHH and IHH ligands¹⁶ and blocks signaling by disrupting ligand binding to PTCH¹⁷. Autonomous activation of transfected reporter was not affected by control antibody, but was dramatically reduced by incubation with 5E1 at 0.1 or 10 μg/ml (FIG. 4A). Reporter activity in contrast was augmented approximately 8 fold by addition of purified SHH ligand to 25 nM (FIG. 4A). Addition of 5E1 in combination with SHH ligand reduced reporter activity to a level intermediate between those seen with either reagent alone (FIG. 4A), indicating a mutual antagonism between 5E1 and ligand in activation of pathway.

Reporter activity in cells from single passage pancreatic cancer xenografts was also antagonized by 5E1 (FIG. 4B). Treatment with 5E1 antibody dramatically reduced viable cell mass as well (FIG. 4C), and both the cell-killing effect and reporter effect were observed exclusively in cells from tumors that expressed endogenous PTCH mRNA. The relationship between ligand concentration and growth was further investigated by adding 5E1 antibody to cells from a single passage pancreatic tumor xenograft at a level just sufficient to block growth, then adding SHH protein. Growth correlated positively with increasing concentrations (FIG. 4D). Rates of growth from this experiment plotted as a function of SHH concentration (FIG. 4E) indicate that ligand-induced pathway activation is rate limiting and that unperturbed growth of these cells is sub-maximal.

Hh ligand and 5E1 blocking antibody were mutually antagonistic in their effects on reporter activity, and produced opposite effects on growth of cells from these gut-derived tumors (FIGS. 4A to 4E), thus revealing a Hh ligand-dependent mechanism for pathway activation and cell growth. In contrast, addition of Hh ligand or of 5E1 blocking antibody did not significantly affect growth of cells from a single passage pancreatic tumor xenograft that did not express PTCH mRNA (FIG. 4F), demonstrating the specificity of antibody and ligand effects. No significant ligand-induced or antibody-induced change in growth was observed in medulloblastoma cells derived from a mouse model of Gorlin syndrome (FIG. 4F), in which the Hh pathway is activated through loss of PTCH function^{14,18}. However, in contrast to the antibody-resistant xenograft cells, the medulloblastoma-derived cells require pathway activity for growth and can be killed by cyclopamine treatment¹⁴.

Ligand-independent mutational activation of the Hh pathway has been linked to the formation of tumors associated with Gorlin Syndrome (e.g., medulloblastoma). Despite a widespread activation of and dependence on the Hh pathway for medulloblastoma growth¹⁴, however, only a fraction of sporadic tumors can be assigned to pathway-activating mutations, suggesting that other mechanisms of pathway activation are involved. The present Example demonstrates that Hh pathway activation and growth of cells from a group of gut-derived malignancies is ligand-dependent. Small cell lung cancer (SCLC), also arising from endodermal derived epithelium and associated with Hh ligand expression, recently has been linked to transient reactivation of the Hh pathway within the airway epithelium for regulation of progenitor cell fates during injury repair⁷. A similar role for Hh signaling in renewal of the epithelium of the gut and its derivatives is suggested by embryonic and adult expression of the Hh pathway targets PTCH and GLI (see, e.g., Ref. 9) and by the requirement for Hh signaling for stem cell proliferation within the murine gut epithelium⁹.

It is not known whether renewal of injured gut epithelium is associated with transient Hh pathway reactivation, but it is notable that increased rates of esophageal, gastric, and pancreatic carcinomas occur in association with acid injury in Barrett esophagus, in Helicobacter pylori infection, and upon exposure to alcohol, cigarette smoke, and certain dietary components⁹⁻²¹. Exposure to such factors likely causes injury to the gut epithelium, eliciting a chronic state of injury repair and a consequent increase in proliferative stem or progenitor cells that may arise through ligand-dependent reactivation of the Hh pathway. Many of these agents are also mutagenic, thus potentially enhancing tumor formation by subjecting an enlarged pool of stem or stem-like target cells to potentially oncogenic mutations. However induced, the present results identify a group of common and frequently lethal gut-derived tumors, which can be readily diagnosed by their expression of endogenous pathway targets such as PTCH, that can be treated using antagonist-mediated or antibody-mediated pathway blockade, even in advanced stages of metastatic disease.

### REFERENCES

Each of the following publications is incorporated herein by reference.
1. Wechsler-Reya and Scott, The developmental biology of brain tumors. Ann. Rev. Neurosci. 24, 385-428 (2001).
2. Bale and Yu, The hedgehog pathway and basal cell carcinomas. Hum. Mol. Genet. 10, 757-62. (2001).
3. Taipale et al., Patched acts catalytically to suppress the activity of Smoothened. Nature 418, 892-7 (2002).
4. Ingham and McMahon, Hedgehog signaling in animal development: paradigms and principles. Genes Dev 15, 3059-87 (2001).
5. Taipale and Beachy, The Hedgehog and Wnt signaling pathways in cancer. Nature 411, 349-54. (2001).
6. Chen et al., Inhibition of Hedgehog signaling by direct binding of cyclopamine to Smoothened. Genes Dev 16, 2743-8 (2002).
7. Watkins et al., Hedgehog signaling within airway epithelial progenitors and in small-cell lung cancer. Nature 422, 313-7 (2003).
8. Hebrok, M. Hedgehog signaling in pancreas development. Mech Dev 120, 45-57 (2003).
9. Ramalho-Santos et al., Hedgehog signals regulate multiple aspects of gastrointestinal development. Development 127, 2763-72 (2000).
10. Roberts et al., Epithelial-mesenchymal signaling during the regionalization of the chick gut. Development 125, 2791-801 (1998).
11. Bitgood and McMahon, Hedgehog and Bmp genes are coexpressed at many diverse sites of cell-cell interaction in the mouse embryo. Dev Biol 172, 126-38 (1995).
12. Taipale et al., Effects of oncogenic mutations in Smoothened and Patched can be reversed by cyclopamine. Nature 406, 1005-9 (2000).
13. Cooper et al., Plant-derived and synthetic teratogens inhibit the ability of target tissues to respond to Sonic hedgehog signaling. Science 280, 1603-1607 (1998).
14. Berman et al., Medulloblastoma growth inhibition by hedgehog pathway blockade. Science 297, 1559-61 (2002).
15. Ericson et al., Two critical periods of Sonic Hedgehog signaling required for the specification of motor neuron identity. Cell 87, 661-73 (1996).
16. Wang et al., Regular articles: conditional disruption of hedgehog signaling pathway defines its critical role in hair development and regeneration. J Invest Dermatol. 114, 901-8 (2000).
17. Fuse et al., Sonic hedgehog protein signals not as a hydrolytic enzyme but as an apparent ligand for patched. Proc Natl Acad Sci USA 96, 10992-9 (1999).
18. Goodrich et al., Altered neural cell fates and medulloblastoma in mouse patched mutants. Science 277, 1109-1113 (1997).
19. Chen and Yang, Esophageal adenocarcinoma: a review and perspectives on the mechanism of carcinogenesis and chemoprevention. Carcinogenesis 22, 1119-29 (2001).
20. Peek, R. M., Jr., Helicobacter pylori strain-specific modulation of gastric mucosal cellular turnover: implications for carcinogenesis. J Gastroenterol 37 Suppl 13, 10-16 (2002).
21. Lowenfels and Maisonneuve, Epidemiologic and etiologic factors of pancreatic cancer. Hematol Oncol Clin North Am 16, 1-16 (2002).
22. Chen et al., Small molecule modulation of Smoothened activity. Proc Natl Acad Sci USA99, 14071-6 (2002).

TABLE

| | | | | | | REF./ |
|---|---|---|---|---|---|---|
| DESIGNATION | TUMOR | HISTOLOGY | GRADE | Sample type | Stage | SOURCE |
| SEG1 | ESOPHAGUS | ADENOCA | — | Cell Line | Pri. | S1 |
| OE33 | ESOPHAGUS | ADENOCA | — | Cell Line | Pri. | S2 |
| KYAE | ESOPHAGUS | ADENOCA | — | Cell Line | Pri. | S3 |
| KYSE70 | ESOPHAGUS | SQUAMOUS | — | Cell Line | Pri. | S3 |
| KYSE510 | ESOPHAGUS | SQUAMOUS | — | Cell Line | Pri. | S3 |
| KYSE180 | ESOPHAGUS | SQUAMOUS | — | Cell Line | Pri. | S3 |
| KYSE150 | ESOPHAGUS | SQUAMOUS | — | Cell Line | Pri. | S3 |
| NCI-SNU1 | STOMACH | ADENOCA | High (Signet ring) | Cell Line | Pri. | S4 |
| NCI-SNU16 | STOMACH | ADENOCA | High (Signet ring) | Cell Line | Pri. | S4 |
| NCI-N-87 | STOMACH | ADENOCA | Low | Cell Line | Met. | S4 |
| RF1# | STOMACH | ADENOCA | High (Signet ring) | Cell Line | Pri. | S4 |
| RF48# | STOMACH | ADENOCA | High (Signet ring) | Cell Line | Met. | S4 |
| AGS | STOMACH | ADENOCA | Moderate | Cell Line | Pri. | S4 |
| PrGas 1 | STOMACH | ADENOCA | Moderate | Snap-frozen | Pri. | S5 |
| PrGas 2 | STOMACH | ADENOCA | High (Signet ring) | Snap-frozen | Pri. | S5 |
| PrGas 3 | STOMACH | ADENOCA | High (Signet ring) | Snap-frozen | Pri. | S5 |
| PrGas 4 | STOMACH | ADENOCA | Moderate | Snap-frozen | Pri. | S5 |
| PrGas 5 | STOMACH | ADENOCA | High (Signet ring) | Snap-frozen | Pri. | S5 |
| PrGas 6 | STOMACH | ADENOCA | High (Signet ring) | Snap-frozen | Met. | S5 |
| PrGas 7 | STOMACH | ADENOCA | High (Signet ring) | Snap-frozen | Pri. | S5 |
| PrGas 8 | STOMACH | ADENOCA | High (Signet ring) | Snap-frozen | Pri. | S5 |
| PrGas 9 | STOMACH | ADENOCA | Moderate | Snap-frozen | Pri. | S5 |
| PrGas 10 | STOMACH | ADENOCA | High (Signet ring) | Snap-frozen | Pri. | S5 |
| SNU308 | GALLBLADDER | ADENOCA | Moderate | Cell Line | Pri. | S6 |
| SNU1079 | BILE DUCT | ADENOCA | Moderate | Cell Line | Pri. | S6 |
| SNU245 | BILE DUCT | ADENOCA | Moderate | Cell Line | Pri. | S6 |
| HUCCT1 | BILE DUCT | ADENOCA | High | Cell Line | Met. | S7 |
| TFK1 | BILE DUCT | ADENOCA | Moderate | Cell Line | Pri. | S8 |
| GBD1 | GALLBLADDER | ADENOCA | — | Cell Line | Met. | S9 |
| G415 | GALLBLADDER | ADENOCA | — | Cell Line | Pri. | S10 |
| GBH3 | GALLBLADDER | ADENOCA | — | Cell Line | Pri. | S11 |
| GBK1 | GALLBLADDER | ADENOCA | — | Cell Line | Pri. | S11 |
| PANC1 | PANCREAS | ADENOCA | — | Cell Line | Pri. | S4 |
| HS766T | PANCREAS | ADENOCA | — | Cell Line | Met. | S4 |
| PL6 | PANCREAS | ADENOCA | — | Cell Line | Pri. | S12 |
| PL5 | PANCREAS | ADENOCA | — | Cell Line | Pri. | S12 |
| BXPC3 | PANCREAS | ADENOCA | — | Cell Line | Pri. | S4 |
| CFPAC1 | PANCREAS | ADENOCA | — | Cell Line | Pri. | S4 |
| PX154 | PANCREAS | ADENOCA | Moderate | Xenograft | Pri. | S5 |
| PX155 | PANCREAS | ADENOCA | Moderate | Xenograft | Met. | S5 |
| PX169 | PANCREAS | ADENOCA | High | Xenograft | Pri. | S5 |
| PX183 | PANCREAS | ADENOCA | Moderate | Xenograft | Pri. | S5 |
| PX184 | PANCREAS | ADENOCA | Moderate | Xenograft | Pri. | S5 |
| PX185 | PANCREAS | ADENOCA | High | Xenograft | Pri. | S5 |
| PX196 | PANCREAS | ADENOCA | High | Xenograft | Pri. | S5 |
| PrPanc 1 | PANCREAS | ADENOCA | High | Snap-frozen | Pri. | S5 |
| PrPanc 2 | PANCREAS | ADENOCA | High | Snap-frozen | Pri. | S5 |
| PrPanc 3 | PANCREAS | ADENOCA | High | Snap-frozen | Pri. | S5 |
| PrPanc 4 | PANCREAS | ADENOCA | Moderate | Snap-frozen | Pri. | S5 |
| PrPanc 5 | PANCREAS | ADENOCA | High | Snap-frozen | Pri. | S5 |
| PrPanc 6 | PANCREAS | ADENOCA | Moderate | Snap-frozen | Pri. | S5 |
| PrPanc 7 | PANCREAS | ADENOCA | High | Snap-frozen | Pri. | S5 |
| PrPanc 8 | PANCREAS | ADENOCA | High | Snap-frozen | Pri. | S5 |
| PrPanc 9 | PANCREAS | ADENOCA | High | Snap-frozen | Pri. | S5 |
| PrPanc 10 | PANCREAS | ADENOCA | Moderate | Snap-frozen | Pri. | S5 |
| PrPanc 11 | PANCREAS | ADENOCA | High | Snap-frozen | Pri. | S5 |
| PrPanc 12 | PANCREAS | ADENOCA | Moderate | Snap-frozen | Pri. | S5 |
| PrPanc 13 | PANCREAS | ADENOCA | Moderate | Snap-frozen | Pri. | S5 |
| PrPanc 14 | PANCREAS | ADENOCA | High | Snap-frozen | Pri. | S5 |
| PrPanc 15 | PANCREAS | ADENOCA | High | Snap-frozen | Pri. | S5 |
| SKCO1 | COLON | ADENOCA | — | Cell Line | Pri. | S4 |
| D2D1 | COLON | ADENOCA | — | Cell Line | Pri. | S4 |
| HCT116 | COLON | ADENOCA | — | Cell Line | Pri. | S4, S14 |
| HT29 | COLON | ADENOCA | — | Cell Line | Pri. | S4 |
| SW1417 | COLON | ADENOCA | — | Cell Line | Pri. | S4 |
| SW837 | COLON | ADENOCA | — | Cell Line | Pri. | S4 |
| COLO205 | COLON | ADENOCA | — | Cell Line | Pri. | S4 |
| RKO | COLON | ADENOCA | — | Cell Line | Pri. | S4 |
| SW948 | COLON | ADENOCA | — | Cell Line | Pri. | S4 |
| LOVO | COLON | ADENOCA | — | Cell Line | Pri. | S4 |
| PZp53-MED1 | CEREBELLUM | MEDULLO. | — | Cell Line | Pri. | S13 |

ADENOCA, adenocarcinoma;
Pri., primary;
Met., metastasis.

HuCCT1 and NCI-N-87 were established from ascitic fluid (i.e., metastatic cholangiocarcinoma and gastric ADENOCA, respectively); GBD1 and HS766T were established from nodal metastases of a gallbladder and pancreatic ADENOCA, respectively. The remaining cell lines/xenografts are established from primary tumors, with the exceptions listed below: RF-48 was derived from the ascitic fluid (i.e., metastatic gastric ADENOCA) of the same patient from whom RF-1 was derived; PX155 was established from a lymph node Met. arising from the same patient from whom PX154 is derived. HCT116 and HCT116+ch3 (FIG. 1) are isogenic colon cancer cell lines except that the latter contains an extra copy of chromosome3¹⁴.
SOURCE REFERENCES in Table, each of which is incorporated herein by reference: S1) Soldes et al., Br. J. Cancer, 79: 595-603, 1999; S2) Rockett et al., Br J. Cancer. 75(2):258-63, 1997; S3) Shimada et al., Cancer 69(2):277-84, 1992; S4) see American Type Culture Collection (at URL “www.atcc.org”); S5) Surgical material from The Johns Hopkins Hospital collected in accordance to institutionally approved protocols; S6) Ku et al., Br J Cancer 87(2): 187-93, 2002; S7) Miyagiwa et al., In Vitro Cell. Dev. Biol. 25, pp. 503-510, 1989); S8) Saijyo et al., Tohoku J Exp Med. 177(1):61-71, 1995; S9) Shimura et al., Jpn J Cancer Res. 186(7):662-9, 1996; S10) Koyama et al., Gann. 1980 August; 71(4):574-5, 1980; S11) Li et al., Clin Exp Met. 16(1):74-82, 1988; S12) Jaffee et al., Cancer J Sci Am 4(3): 194-203, 1998; S13) Berman et al., Science 297:1559-1561, 2002; S14) Boland, C R. Int J Cancer 69:47-9; 1996.

Although the invention has been described with reference to the above example, it will be understood that modifications and variations are encompassed within the spirit and scope of the invention. Accordingly, the invention is limited only by the following claims.

What is claimed is:

1. A method of reducing or inhibiting proliferation of cells of a digestive tract tumor characterized by abnormally elevated Hedgehog (Hh) pathway activity, comprising contacting the cells with at least one Hh pathway antagonist, thereby reducing or inhibiting proliferation of the cells of the digestive tract tumor.

2. The method of claim 1, wherein the digestive tract tumor is a malignant tumor.

3. The method of claim 2, wherein the cells are pancreatic cancer cells, stomach cancer cells, esophagus cancer cells, or biliary tract cancer cells.

4. The method of claim 2, wherein the cells are colon cancer cells.

5. The method of claim 1, wherein the abnormally elevated Hh pathway activity comprises abnormally elevated ligand stimulated Hh pathway activity.

6. The method of claim 5, wherein the ligand comprises Sonic hedgehog (SHH) or Indian hedgehog (IHH).

7. The method of claim 1, wherein the Hh pathway antagonist comprises a peptide, a polynucleotide, a peptidomimetic, or a small organic molecule.

8. The method of claim 1, wherein the Hh pathway antagonist comprises an anti-Hh antibody.

9. The method of claim 6, wherein the anti-Hh antibody comprises an anti-SHH antibody, an anti-IHH antibody, or an anti-SHH antibody and an anti-IHH antibody.

10. The method of claim 1, wherein the Hh pathway antagonist comprises a steroidal alkaloid or a derivative thereof.

11. The method of claim 10, wherein Hh pathway antagonist comprises cyclopamine.

12. A method of ameliorating a digestive tract tumor comprising cells characterized by abnormally elevated Hedgehog (Hh) pathway activity in a subject, comprising administering to the subject an Hh pathway antagonist, whereby the Hh pathway antagonist contacts cells of the tumor in the subject, thereby ameliorating the digestive tract tumor in the subject.

13. The method of claim 12, wherein the digestive tract tumor is a malignant tumor.

14. The method of claim 13, wherein the cells are pancreatic cancer cells, stomach cancer cells, esophagus cancer cells, or biliary tract cancer cells.

15. The method of claim 13, wherein the cells are colon cancer cells.

16. The method of claim 12, wherein the abnormally elevated Hh pathway activity comprises abnormally elevated ligand stimulated Hh pathway activity.

17. The method of claim 12, wherein the Hh pathway antagonist is administered orally.

18. The method of claim 12, wherein the Hh pathway antagonist comprises a peptide, a polynucleotide, a peptidomimetic, or a small organic molecule.

19. The method of claim 12, wherein the Hh pathway antagonist comprises an antibody.

20. The method of claim 19, wherein the antibody comprises an anti-Sonic hedgehog antibody, an anti-Indian hedgehog antibody, or a combination thereof.

21. The method of claim 12, wherein the Hh pathway antagonist comprises a steroidal alkaloid or a derivative thereof.

22. The method of claim 21, wherein Hh pathway antagonist comprises cyclopamine.

23. A method of identifying a digestive tract tumor of a subject amenable to treatment with a Hedgehog (Hh) pathway antagonist, comprising detecting abnormally elevated Hh pathway activity in a sample of cells of the digestive tract tumor of the subject as compared to Hh pathway activity in corresponding normal cells, thereby identifying a digestive tract tumor of a subject amenable to treatment with an Hh pathway antagonist.

24. The method of claim 23, wherein the abnormally elevated Hh pathway activity comprises ligand stimulated Hh pathway activity.

25. The method of claim 23, comprising detecting abnormally elevated expression of at least one Hh pathway polypeptide.

26. The method of claim 25, wherein the Hh pathway polypeptide comprises an Hh ligand, an Hh ligand receptor, or a transcription factor.

27. The method of claim 26, wherein the Hh ligand comprises Sonic hedgehog (SHH), Indian hedgehog (IHH), or SHH and IHH.

28. The method of claim 26, wherein the Hh ligand receptor comprises Patched.

29. The method of claim 26, wherein the transcription factor comprises a GLI-1 transcription factor.

30. The method of claim 25, which comprises detecting elevated levels of a polynucleotide encoding the Hh pathway polypeptide.

31. The method of claim 30, wherein the polynucleotide comprises RNA.

32. The method of claim 31, which comprises performing a reverse transcription-polymerase chain reaction.

33. The method of claim 25, which comprises detecting elevated levels of the Hh pathway polypeptide.

34. The method of claim 33, which comprises performing an immunoassay.

35. The method of claim 23, comprising detecting abnormally elevated activity of the Hh pathway polypeptide.

36. The method of claim 35, wherein the Hh pathway polypeptide comprises a transcription factor.

37. The method of claim 46, which comprises detecting increased binding activity of the transcription factor to a cognate transcription factor regulatory element.

38. The method of claim 36, which comprises detecting increased expression of a reporter gene comprising a cognate transcription factor regulatory element.

39. The method of claim 25, which comprises detecting altered expression of a transcriptional target of the Hh pathway.

40. The method of claim 39, wherein the transcriptional target comprises a nestin gene or a BMI-1 gene.

41. The method of claim 39, which comprises detecting increased expression of a gene that is positively regulated by GLI-1 or GLI-2.

42. The method of claim 39, which comprises detecting decreased expression of gene that is negatively regulated by GLI-3.

43. The method of claim 23, comprising detecting abnormally decreased expression of at least one Hh pathway polypeptide.

44. The method of claim 43, wherein the Hh pathway polypeptide comprises a Gli-3 transcription factor.

45. The method of claim 23, wherein the sample comprises a biopsy sample obtained from the subject.

46. The method of claim 23, further comprising contacting cells of the sample with at least one Hh pathway antagonist, and detecting a decrease in Hh pathway activity in the cells following said contact, thereby confirming that the digestive tract tumor is amenable to treatment with an Hh pathway antagonist.

47. A method of identifying an agent useful for treating a digestive tract tumor having abnormally elevated Hedgehog (Hh) pathway activity, comprising contacting a sample of cells of a digestive tract tumor with at least one test agent, wherein a decrease in Hh pathway activity in the presence of the test agent as compared to Hh pathway activity in the absence of the test agent identifies the agent as useful for treating the digestive tract tumor.

48. The method of claim 47, wherein the abnormally elevated Hh pathway activity comprises abnormally elevated ligand stimulated Hh pathway activity.

49. The method of claim 47, wherein the agent comprises a peptide, a polynucleotide, a peptidomimetic, or a small organic molecule.

50. The method of claim 47, wherein the agent comprises an Hh pathway antagonist.

51. The method of claim 50, wherein the Hh pathway antagonist comprises an antibody.

52. The method of claim 51, wherein the antibody comprises an anti-Sonic hedgehog antibody, an anti-Indian hedgehog antibody, or a combination thereof.

53. The method of claim 50, wherein the Hh pathway antagonist comprises a steroidal alkaloid or a derivative thereof.

54. The method of claim 53, wherein Hh pathway antagonist comprises cyclopamine.

55. The method of claim 50, wherein the Hh pathway antagonist comprises a Smoothened antagonist.

56. The method of claim 47, wherein the sample of cells of the digestive tract tumor is obtained from a subject having the digestive tract tumor.

57. The method of claim 56, wherein the sample of cells is obtained by biopsy.

58. The method of claim 56, wherein the digestive tract tumor comprises a malignant tumor.

59. The method of claim 58, wherein the cells are pancreatic cancer cells, stomach cancer cells, esophagus cancer cells, or biliary tract cancer cells.

60. The method of claim 58, wherein the cells are colon cancer cells.

61. The method of claim 47, which said contacting comprises contacting the sample of cells in culture.

62. The method of claim 47, which is performed in a high throughput format.

63. The method of claim 62, comprising contacting samples of cells of a plurality of samples with at least one test agent.

64. The method of claim 63, wherein samples of cells of the plurality are obtained from a single subject.

65. The method of claim 64, comprising contacting different samples of cells of the plurality with same amounts of a test agent, with different amounts of a test agent, with same amounts of different test agents, with different amounts of different test agents, or a combination thereof.

66. The method of claim 63, wherein samples of cells of the plurality are obtained from different subjects.

67. The method of claim 46, comprising contacting the cells with at least two test agents.
